(19) United States
(12) Patent Application Publication
(10) Pub. No.: US 2006/0014282 A1
(43) Pub. Date: Jan. 19, 2006

(54) Cell and tissue engineering utilizing LIF

(75) Inventors: Nicolas Fortunel, Breuillet (FR); Dominique Bernard, Paris (FR); Corinne Ferraris, Paris (FR); Marcelle Regnier, Paris (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: L'OREAL, PARIS (FR)

(21) Appl. No.: 11/137,402

(22) Filed: May 26, 2005

Related U.S. Application Data
(60) Provisional application No. 60/641,435, filed on Jan. 6, 2005.

(30) Foreign Application Priority Data
May 26, 2004 (FR) 04/51037

(51) Int. Cl. C12N 5/08

(52) U.S. Cl. 435/371

(57) ABSTRACT

Leukemia Inhibitory Factor (“LIF”), an LIF analogue, an LIF mimetic and a product capable of stimulating the expression of endogenous LIF, and mixtures thereof are useful, for (i) promoting the multiplication, in vitro, of a population of human skin stem cells and/or epidermal progenitors while maintaining them in an undifferentiated state, and/or for (ii) maintaining and/or increasing their capacity to generate a pluristratified epithelium, in particular a pluristratified epidermis and/or all or some of the skin appendages; also a library or a culture of human undifferentiated skin stem cells and/or epidermal progenitors is obtained in the presence of LIF, as are reconstructed epidermides and/or reconstructed skin, and kits for producing libraries of cells or reconstructed epidermides, and stem cells or reconstructed epidermides are prepared for the treatment of individuals exhibiting damaged skin (individuals suffering from third degree burns and individuals suffering from genetic diseases affecting the skin).

### CROSS-REFERENCE TO PRIORITY/PROVISIONAL APPLICATIONS

This application claims priority under 35 U.S.C. § 119 of FR 04/51037, filed May 26, 2004, and of provisional application Serial No. 60/641,435, filed Jan. 6, 2005, each hereby expressly incorporated by reference and each assigned to the assignee hereof. This application is also a continuation of said '435 provisional.

### CROSS-REFERENCE TO COMPANION APPLICATION

Our copending application Ser. No. ______ [Attorney Docket No. 016800-738], filed concurrently herewith and also assigned to the assignee hereof.

### BACKGROUND OF THE INVENTION

### TECHNICAL FIELD OF THE INVENTION

The present invention relates to methods for culturing human skin stem cells and/or epidermal progenitors and their uses especially for preparing pluristratified epithelia, in particular epidermis equivalents and/or skin equivalents.

The present invention also relates, in particular, to the use of an effective amount of a compound selected from among LIF, a LIF analogue, a LIF mimetic and a product capable of stimulating the endogenous expression of LIF, and mixtures thereof, for (i) promoting the multiplication, in vitro, of a population of human skin stem cells and/or epidermal progenitors while maintaining them in an undifferentiated state and/or for (ii) maintaining and/or increasing their capacity to generate a pluristratified epithelium, in particular a pluristratified epidermis, and/or all or some of the skin appendages.

This invention also relates to a method for obtaining a library or a culture of human undifferentiated skin stem cells and/or epidermal progenitors in the presence of LIF, to a method for obtaining reconstructed epidermides and/or reconstructed skin, to kits for producing libraries of cells or reconstructed epidermides, and to the uses of LIF, in particular for preparing stem cells or reconstructed epidermides suited for the treatment of individuals exhibiting damaged skin (individuals with third degree burns and individuals suffering from genetic diseases affecting the skin).

According to the invention, the expression “population of human undifferentiated skin stem cells and/or epidermal progenitors” means a population of adult somatic stem cells naturally present in the basal layer of the epidermis, which are capable of self-renewal and/or of proliferating, and of generating a pluristratified epithelium, in particular a pluristratified epidermis, and/or all or some of the skin appendages (sebaceous glands, hair follicle, nail, etc.). This population of human undifferentiated skin stem cells and/or epidermal progenitors may, for example, be obtained from samples of adult skin and/or of neonatal skin.

The expression “capacity of a cell for self-renewal” means a cell capable of dividing so as to give two daughter cells, at least one of which is identical to the mother cell. On the scale of a complex cell population, the notion of self-renewal implies the maintenance of a compartment of cells having phenotypic and functional characteristics that are constant in the course of the successive cell divisions. According to the invention, this involves the maintenance of a compartment made up of undifferentiated skin stem cells and/or epidermal progenitors, in particular characterized by a strong proliferative potential and a capacity to generate a pluristratified epithelium, in particular a pluristratified epidermis.

The expression “capacity of a cell to proliferate” means a cell capable of multiplying so as to give two daughter cells, without their necessarily being transmission of the characteristics and of the potential of the mother cell to at least one of the two daughter cells.

The proliferation, which may or may not be associated with the self-renewal phenomenon, is liable to result in the gradual decrease in or the disappearance of the cell compartment of interest within the cell population that multiplies.

According to the invention, the cell proliferation may be accompanied by a gradual decrease in and/or by the disappearance of the compartment of undifferentiated skin stem cells and/or epidermal progenitors, in particular characterized by a strong proliferative potential and a capacity to generate a pluristratified epithelium, in particular a pluristratified epidermis.

This population comprises in particular undifferentiated skin stem cells and/or epidermal progenitors.

The stem cell compartment, located most upstream in the skin tissue hierarchy, is characterized by a strong potential for self-renewal (greatest long-term multiplication capacity), and also by a multipotentiality (especially a capacity to produce a pluristratified epithelium, in particular keratinocytes and/or various cell types of the skin appendages, such as hair follicles, sebaceous glands, nails, etc.).

The epidermal progenitors derived from said stem cells exhibit a multiplication potential which can also be considerable, but is generally less than that of the stem cells, and are characterized by a capacity to generate a pluristratified epithelium, in particular a pluristratified epidermis.

Stem cells of embryonic origin (ES cells) have a potential for expansion that is a priori unlimited (Amit et al., Dev. Biol., 227: 271-278, 2000; Odorico et al., Stem Cells., 19: 193-204, 2001). In fact, they can be multiplied without signs of aging while at the same time conserving an undifferentiated state.

WO 02/097068 describes in particular a method for inducing the differentiation of murine embryonic stem cells to murine keratinocytes.

On the other hand, the potential for expansion of adult somatic stem cells is limited by a mitotic clock (Vaziri et al., Proc. Natl. Acad. Sci. USA., 91: 9857-9860, 1994; Chang et al., Proc. Natl. Acad. Sci. USA., 92: 11190-11194, 1995). This limited expansion capacity concerns in particular epidermal stem cells and progenitors (Ramirez et al., Genes Dev., 15: 398-403, 2001; Counter et al., Lancet., 361: 1345-1346, 2003; Fortunel et al., J. Cell Sci., 116: 4043-4052, 2003).

After a limited number of divisions, stem cells and progenitors derived from adult tissues invariably become oriented towards differentiation, exhibit signs of senescence, and die. These characteristics represent limiting factors for the production of cell libraries and for cell and tissue engineering (for example: preparation of skin equivalents).

Previously proposed, in WO 03/038073 (L'OREAL/CNRS), was a method for enriching a population of keratinocyte stem cells from a keratinocyte preparation, by adhesion of the preparation to an extracellular matrix component.

Cell culture methods, in particular methods for multiplying and/or differentiating cells, should therefore take into account the characteristics specific to adult somatic stem cells and to embryonic stem cells, which differ in particular through (i) their potential for expansion, acknowledged to be unlimited for embryonic stem cells and limited for adult stem cells; and (ii) their stage of differentiation, which is more advanced for skin stem cells and/or epidermal progenitors (capable of generating skin tissues), compared with “totipotent” embryonic stem cells (capable of generating all the tissue and organ types of the individual).

Need continues to exist for agents capable of promoting the multiplication of human skin stem cells and/or epidermal progenitors while maintaining them in an undifferentiated state, and which are capable of maintaining, or even increasing, their organogenic potential, i.e., their capacity to generate a pluristratified epithelium, in particular a pluristratified epidermis and/or all or some of the skin appendages (hair follicle, sebaceous glands, nails, etc.).

### SUMMARY OF THE INVENTION AND DESCRIPTION OF BACKGROUND AND/OR RELATED AND/OR PRIOR ART

It has now unexpectedly been determined that the use of Leukemia Inhibitory Factor (LIF) makes it possible:

(i) to promote the multiplication, in vitro, of a population of human skin stem cells and/or epidermal progenitors while maintaining them in an undifferentiated state;

(ii) and even appears to be necessary for obtaining a pluristratified reconstructed epidermis of quality, i.e., exhibiting histological characteristics comparable to those of a natural epidermis (with a horny layer, a granular layer, suprabasal layers and a basal layer). It has in fact been shown that an epidermis reconstructed in the absence of LIF exhibits structural anomalies compared with an epidermis reconstructed in the presence of LIF.

LIF is described, in U.S. Pat. No. 6,261,548, as a factor capable of suppressing the proliferation of myeloid leukemia cells such as murine M1 cells, and of promoting macrophage differentiation, suggesting its use as a non-proliferative therapeutic agent for suppressing certain forms of myeloid leukemias and for modifying the function of macrophages involved in the response to infections.

In the skin, LIF is a cytokine that is naturally produced by the keratinocytes in vivo and in vitro (Paglia et al., Br. J. Dermatol., 134: 817-823, 1996), and it is known that this factor is in particular involved in the control of inflammatory processes associated with various skin pathologies such as psoriasis (Bonifati et al., Arch. Dermatol. Res., 290: 9-13, 1998; Szepietowski et al., J. Dermatol., 28: 115-122, 2001), or certain allergies (Szepietowski et al., Contact Dermatitis., 36: 21-25, 1997).

However, the use of LIF, of a LIF analogue, of a LIF mimetic or of a product capable of stimulating the expression of endogenous LIF, or mixtures thereof, for promoting the multiplication, in vitro, of adult somatic stem cells, known to have a limited expansion potential compared with embryonic stem cells, and a more advanced stage of differentiation than the latter, a fortiori the use of LIF for promoting the multiplication, in vitro of human undifferentiated skin stem cells and/or epidermal progenitors, is in no way described or suggested, nor is it for improving organotypic culture models for skin (reconstructed epidermides and reconstructed skin).

The importance of this discovery for obtaining libraries or cultures of human undifferentiated skin stem cells and/or epidermal progenitors, and for preparing skin equivalents intended to treat individuals exhibiting lesioned skin (individuals suffering from third degree burns, individuals suffering from a genetic disease affecting the skin), is apparent.

The skin constitutes a physical barrier between the organism and its environment. It consists of two tissues: the epidermis and the dermis.

The dermis gives the epidermis a solid support. It is also its nourishing factor. It consists mainly of fibroblasts and of an extracellular matrix that is itself composed in particular of collagen, of elastin, of fibronectin and of a substance known as ground substance, these components being essentially synthesized by the fibroblasts. Leukocytes, mast cells or tissue macrophages are also found therein. It also contains blood vessels and nerve fibers.

The epidermis is a desquamating pluristratified epithelium, 100 μm thick, on average, and is conventionally divided up into a basal layer containing skin stem cells, undifferentiated epidermal progenitors, and also cells involved in the maturation/differentiation process, a “spiny” layer consisting of several layers of polyhedral cells placed on the basal cells, a “granular” layer consisting of flattened cells containing cytoplasmic inclusions, keratohyalin granules, and, finally, an upper layer called horny layer (or stratum comeum), consisting of keratinocytes at the terminal stage of their differentiation, called corneocytes. The latter are mummified, anuclear keratinized cells that derive from keratinocytes. The stacking of the corneocytes constitutes the horny layer which performs, inter alia, the barrier function of the epidermis.

Epidermal differentiation follows a process of continuous and oriented maturation in which the basal keratinocytes transform while migrating so as to result in the formation of corneocytes, dead cells that are completely keratinized. This differentiation is the result of perfectly coordinated phenomena which will result in a constant thickness being maintained and will thus ensure the homeostasis of the epidermis. This means that there must be a precise regulation of the number of cells that enter into the differentiation process and of the number of cells that desquamate. In the course of the normal desquamation process, only the most superficial corneocytes detach from the surface of the epidermis.

Due to its exposure, the skin can be subjected to various types of environmental attacks, the most serious of which can result in a fatal outcome. Patients who have suffered severe burns can exhibit skin tissue destruction over more than 80% of the body surface, and it will only be possible to save them if an amount of skin equivalent sufficient to allow regeneration of the skin over all the areas destroyed is successfully produced.

The control of the expansion potential of skin stem cells and/or of epidermal progenitors and the development of organotypic skin cultures from cells amplified ex vivo are therefore questions of major importance for the production of autologous (Ronfard et al., Burns., 17: 181-184, 1991 and Transplantation., 70: 1588-1598, 2000) and/or heterologous (Hefton et al., Lancet., 2: 428-430, 1983; Braye et al., Med. Biol. Eng. ComPut., 38: 248-252, 2000) skin tissue grafts intended for the treatment of individuals suffering from third degree burns.

Epidermis equivalents produced from cells, derived from skin explants and amplified ex vivo, are also used to produce autologous grafts for the regeneration of skin tissue after surgical excision of a naevus (Kumagai et al., Ann. Plast. Surg., 39: 483-488, 1997) or of a tattoo (Kumagai et al., Ann. Plast. Surg., 33: 385-391, 1994).

The skin can also be affected by various diseases of genetic origin that compromise its integrity. These are, for example, epidermolysis bullosa, characterized by a permanent deficiency in cohesion between the dermis and the epidermis (Eady et al., J. Dernatol., 28: 638-640, 2001); ichthyoses, associated with a pathological thickening of the horny layer of the epidermis (DiGiovanna & Robinson-Bostom, Am. J. Clin. Dermatol., 4: 81-95, 2003).

Other types of genetic diseases that affect the skin, such as Xeroderma pigmentosum, result in an impairment of the capacity of this tissue to respond to “attacks” from the environment. These diseases are characterized by a deficiency of the mechanisms of excision of DNA damage induced by ultraviolet (UV) rays, the clinical consequence of which is an increase in the frequency of appearance of skin cancers (Sarasin, Mutat. Res., 428: 5-10, 1999).

They are therefore serious pathologies, for which no effective conventional pharmacological treatment is currently available. However, the monogenic origin identified for some of these diseases opens up the perspective of treatments by gene therapy (Spirito et al., J. Gen. Med., 3: 21-31, 2001; Magnaldo & Sarasin, Mutat Res., 509: 211-220, 2002).

In fact, restoration of the defective function has been obtained on keratinocytes derived from skin samples taken from affected individuals, cultured ex vivo, and corrected by gene transfer using a retroviral vector, in the case of several genetic diseases of the skin. Through this type of approach, it has in particular been possible to obtain genetic correction of keratinocytes affected by lamellar ichthyoses (Choate et al., Hum. Gene Ther., 7: 2247-2253, 1996), by X-chromosome-linked ichthyoses (Freiberg et al., Hum. Mol. Genet., 6: 927-933, 1997) and also for various groups of Xeroderma pigmentosum (Carreau et al., Hum. Gene Ther., 6: 1307-1315, 1995; Arnaudeau-Begard et al., Hum. Gene Ther., 14: 983-996, 2003).

The importance of having culture systems for “manipulating”, ex vivo, skin stem cells and/or epidermal progenitors derived from normal individuals and/or from patients suffering from genetic diseases affecting the skin is therefore also apparent, as is that of obtaining epidermis equivalents and/or skin equivalents that are as physiological as possible, from these cells.

### DETAILED DESCRIPTION OF BEST MODE AND SPECIFIC/PREFERRED EMBODIMENTS OF THE INVENTION

The present invention therefore features the use of an effective amount of a compound selected from among Leukemia Inhibitory Factor (LIF), a LIF analogue, a LIF mimetic and a product capable of stimulating the expression of endogenous LIF, and mixtures thereof, for (i) promoting the multiplication, in vitro, of a population of human skin stem cells and/or epidermal progenitors while maintaining them in an undifferentiated state, and/or for (ii) maintaining and/or increasing their capacity to generate a pluristratified epithelium.

In particular, this invention features the use of an effective amount of a compound selected from among LIF, a LIF analogue, a LIF mimetic and a product capable of stimulating the expression of endogenous LIF, and mixtures thereof, in a method suitable for obtaining libraries or cultures of human undifferentiated skin stem cells and/or epidermal progenitors.

It also features the use of an effective amount of a compound selected from among LIF, a LIF analogue, a LIF mimetic and a product capable of stimulating the expression of endogenous LIF, and mixtures thereof, in a method suited for obtaining pluristratified epithelia, in particular reconstructed epidermides and/or reconstructed skin.

In the context of the invention, reference will be made to an epidermis equivalent or reconstructed epidermis, and also to a skin equivalent or reconstructed skin.

The action of LIF, of a LIF analogue or a LIF mimetic on the multiplication, in vitro, of a population of human undifferentiated skin stem cells and/or epidermal progenitors, and/or their differentiation and/or the development of a pluristratified epidermis, may be direct or indirect.

It may be direct, through stimulation of the potential for proliferation, for expansion and/or for organogenesis of the skin stem cells and/or epidermal progenitors. It may also be indirect, in particular via factors and/or signals emitted by the LIF-reactive cells in response to this factor, and capable of beneficially stimulating these same cells and/or the neighboring cells. These are, for example, signals transmitted by direct contact between the cells, and/or factors involved in autocrine and/or paracrine regulatory pathways. The control of the expression of the potential of the skin stem cells and/or of the epidermal progenitors involves in particular this type of “dialogue” between the fibroblasts of the dermis and the stem cells and/or the epidermal progenitors. Said cells will, consequently, be more receptive to exogenous stimuli and/or to a signal derived from the environment of the cells.

Epidermal homeostasis results in particular from a finely regulated balance between mitogenic signals that promote cell division and anti-proliferative signals. These signals result in particular from the action of the factors naturally produced by the keratinocytes and/or by the other cell types present in their environment, and in particular secreted by the fibroblasts of the dermis.

As examples of “stimuli and/or signals known to be involved in epidermal homeostasis” mention may in particular be made of:

growth factors, such as the mitogenic growth factors epidermal growth factor (EGF) and keratinocyte growth factor (KGF) (Cook et al., J. Cell Physiol., 146: 277-289, 1991; Andreadis etal., FASEB J., 15: 898-906, 2001; Gamady et al., J. Cell Biochem., 89: 440-449, 2003), or else transforming growth factor-β1 (TGF-β1), a multifunctional growth factor identified in particular for its anti-proliferative effect on keratinocytes in vivo and in vitro (Glick et al., Proc. Natl. Acad. Sci., USA. 90: 6076-6080, 1993; Van Ruissen et al., J. Cell Sci., 107: 2219-2228, 1994; Cui et al., Genes Dev., 9,945-955, 1995). These factors are involved in the highly interactive and interlinked autocrine and paracrine regulatory loops, ensuring both the control of proliferation and that of differentiation (Reiss & Sartorelli, Cancer Res., 47: 6705-6709, 1987; Hertle et al., J. Invest. Dermatol., 104: 260-265, 1995: Edmonson etal., J. Cell Physiol., 179: 201-207, 1999; Yamasaki et al., J. Invest. Dermatol., 120: 1030-1037, 2003; Pasonen-Seppanen et al, J. Invest Dermatol., 120: 1038-1044, 2003), and thus allowing precise regulation of the renewal and/or of the maturation of keratinocytes; and/or

molecules that can complete, modulate and/or interfere with the action of growth factors, such as, for example: vitamin D and its derivatives for their ability to increase keratinocyte sensitivity to the mitogenic action of KGF (Gamady et al., J. Cell Biochem., 89: 440-449, 2003); retinoic acid for its ability to modulate keratinocyte proliferation and/or differentiation (Choi & Fuchs, Cell Regul., 1: 791-809, 1990; Gibbs et al., Arch. Dermatol. Res., 288: 729-738; 1996; Chapellier et al., EMBO J., 21: 3402-3413, 2002), in particular by a mechanism of sensitization of the cells to the action of EGF and of TGF-β (Tong et al., J. Invest. Dermatol., 94: 126-131, 1990).

The “LIF” used according to the invention can in particular be provided in the form of a purified LIF, of a recombinant LIF, of ground cellular material or a cell extract containing LIF, of a cell culture supernatant containing LIF, or mixtures thereof.

A LIF of human origin will preferably be used.

Use may in particular be made of:

a LIF purified from organs, from tissues and/or from cells naturally expressing LIF [for example: pituitary gland cells (Ferrara et al., Proc. Natl. Acad. Sci., USA, 89: 698-702, 1992), dermal fibroblasts (Lorenzo et al., Clin. Immunol. Immunopathol., 70: 260-265, 1994), pulmonary fibroblasts (Elias et al., Am. J. Physiol., 266: L426-435, 1994), placenta and endometrium (Kojima et al., Biol. Reprod., 50: 882-887, 1994), bone marrow stromal cells (Lorgeot et al., Cytokine, 9: 754-758, 1997), kidney cells (Morel et al., Cytokine, 12: 265-271, 2000), cardiomyocytes (Ancey et al. Cytokine, 18: 199-205, 2001)];

a recombinant LIF, as obtained in prokaryotic microorganism (for example: bacterium, E. coli) or eukaryotic cell (for example: yeast, Pichia pastors, Saccharomyces) culture, possibly in the form of a fusion protein (for example: human recombinant LIF sold by Chemicon International Inc.);

a ground material or an extract of cells expressing LIF, in particular a ground material or an extract of nourishing cells (for example: murine fibroblasts of the 3T3 line) expressing LIF, or of cells genetically modified to express LIF, or else of cells stimulated to express LIF;

a cell culture supernatant containing LIF, such as a culture supernatant from 3T3 cells expressing LIF; or mixtures thereof.

As “product capable of stimulating the expression of endogenous LIF”, mention may be made, for example, of IL-1β, a cytokine described for its ability to induce the synthesis and secretion of LIF by fibroblasts, epithelial cells and smooth muscle cells of the human lung (Knight et al., Am. J. Respir. Cell Mol. Biol., 20: 934-841, 1999). Mention may also be made of TNF-α, and also the cyclic AMP analogue 8-bromoadenosine 3′: 5′ monophosphate (8BrcAMP), studied for their ability to induce the LIF promoter in a murine line of cells derived from the medullary stroma (Gollner et al., Cytokine, 11: 656-663, 1999).

According to an alternative, use may be made of any active principle capable, conversely, of repressing the activity of signaling pathways that are antagonists of the anti-differentiating effect of LIF, and in particular that involving the Stat5 protein. These are in particular antisense oligonucleotides, and/or methods of interference via small RNAs (siRNA), or else modulators of kinase and phosphatase activities involved in the activation of Stat molecules.

According to the invention, the term “LIF analogue” means in particular any modified LIF polypeptide or any LIF polypeptide fragment having LIF activity on skin stem cells, and/or epidermal progenitors, i.e., which is capable (i) of promoting the capacity of undifferentiated skin stem cells and/or epidermal progenitors for self-renewal and/or their capacity to proliferate, and (ii) of maintaining and/or increasing their capacity to generate a pluristratified epidermis.

The expression “modified LIF polypeptide having LIF activity” means in particular an LIF polypeptide having undergone one or more modifications, for example so as to increase its stability. The term “modification” means any substitution, deletion and/or insertion of an amino acid or of a small number of amino acids, in particular by substitution of natural amino acids with non-natural amino acids or pseudo amino acids at positions such that the modifications do not significantly affect the biological activity of the LIF.

The modified LIP peptide may be obtained from a human LIF peptide sequence selected from among the GenbankAAA59217 sequence (195 aa), the GenBankAAA51699 sequence (202 aa), and the homologous sequences.

The term “homologous sequence” means a sequence that is identical to at least 70%, preferably at least 85%, and even more preferably at least 95%, of a defined peptide sequence, in the same species or in a different species; this is then referred to as an orthologous peptide sequence.

This modified LIF polypeptide may also be obtained from the sequences of the human LIF gene or cDNA (GenBank M63420, J05436, J03261, X13967), or from the sequences of the murine LIF gene or cDNA (GenBank M63419, J05435, X06381, X12810, S73374), according to conventional cloning and expression techniques.

The expression “LIF polypeptide fragment having LIF activity” means in particular a fragment of a sequence selected from among the GenbankAAA59217 sequence (195 aa), the GenBankAAA51699 sequence (202 aa) and the homologous sequences.

Said fragment will in particular be sufficiently large to reconstitute the tertiary structure of LIF exhibiting the LIF-R-binding and gp130-binding sites.

This polypeptide fragment may also be obtained, according to conventional cloning and expression techniques, from the sequences of the human LIF gene or cDNA (GenBank M63420, J05436, X13967, J03261), or from the sequences of the murine LIF gene or cDNA (GenBank X06381, M63419, J05435, X12810, S73374), and in particular from the coding sequences.

Such LIF analogues that are suitable for implementing the invention may thus be selected according to the method comprising the following steps:

a) a preparation of keratinocytes or of undifferentiated skin stem cells and/or epidermal progenitors is cultured (i) in the presence or (ii) in the absence of the test product;

b) the cells cultured according to (i) and (ii) are studied under a microscope;

c) the capacity of the product to promote the multiplication, in vitro, of the undifferentiated skin stem cells and/or epidermal progenitors is measured by comparing the number of cellular clones in the undifferentiated state obtained (i) in the presence or (ii) in the absence of the test product;

d) the product for which an increased number of cellular clones in the undifferentiated state is obtained in the presence of said product, compared with the number of cellular clones in the undifferentiated state in the absence of said product, is selected;

e) the capacity of said product to regenerate a pluristratified epidermis is then tested according to the following steps:
a. a preparation of keratinocytes is seeded onto a dermal support (i) in the presence or (ii) in the absence of said test product;
b. the structure of the epidermis reconstructed according to (i) and (ii) is observed under a microscope;
c. the product for which an improved structure of the reconstructed epidermis is obtained in the presence of said product, compared with the structure of the epidermis in the absence of said product, is selected.

According to the invention, the term “LIF mimetic” means in particular any agonist of the LIF receptor (LIF-R) alternatively any active agent, or cell extract or fraction of a cell extract capable of activating gp130 and/or the Jak/Stat and Ras/Map kinase signaling pathways, in particular the expression and/or the activity of Stat3.

Use may, for example, be made of antibodies that are LIF receptor agonists, synthetic peptides capable of interacting with LIF receptors and of activating them, or any active agent capable of inducing activation of the signaling pathways involved in the response to LIF, in particular the expression and/or the activity of Stat3, or the activation of Janus-associated tyrosine kinases (JAK).

In particular, use may be made of agents capable of inducing the formation of an LIFR-gp130 heterodimer, such as, for example, the cytokine oncostatin M (OSM), cilliary neurotrohic factor (CNTF) and cardiotrophin-1 (CT-1), which have strong tertiary structure homology with LIF.

Such LIF mimetics suitable for implementing the invention may be selected by means of conventional LIF-R-binding and gp130 activation assays.

These LIF analogues or LIF mimetics may be of natural or synthetic origin.

The term “natural origin” means a compound in the pure state or in solution at various concentrations, obtained by various extraction methods from a tissue (skin, etc.) of natural origin, in particular human epidermis, or from extracts of plant origin.

The term “synthetic origin” means a compound in the pure state or in solution at various concentrations, obtained chemically or by production in an organism after introduction into this organism of the elements required for this production.

It is in fact known that the cellular response to LIF involves a low-affinity receptor (glycoprotein of 190 kD, gp 190) and a high-affinity receptor (glycoprotein of 130 kD, gp 130) (Taupin et al., J. Biol. Chem., 276: 47975-47981, 2001), and, downstream of these receptors, the Jak/Stat and Ras/Map kinase signaling pathways (Ernst et al., J. Biol. Chem., 274: 9729-9737, 1999; Burdon et al., Trends Cell Biol., 12: 432-438, 2002). The Stat3 signal transduction protein plays a predominant role in maintaining the undifferentiated state of murine ES cells in response to LIF (Niwa et al., Genes Dev., 12: 2048-2060, 1998; Matsuda et al., EMBO J., 18: 4261-4269, 1999), whereas the expression of the Stat5 protein is, conversely, associated with these cells entering into differentiation (Nemetz et al., Differentiation, 62: 213-220, 1998).

According to an alternative, a LIF receptor (LIF-R) or a product capable of stimulating the expression of endogenous LIF-R may also be combined with a LIF, a LIF analogue, a LIF mimetic or a product capable of stimulating the expression of LIF.

The present invention also features a method for obtaining a library or a culture of undifferentiated skin stem cells and/or epidermal progenitors, comprising at least one step of multiplication of a preparation of human keratinocytes in a culture medium containing an effective amount of a compound selected from among LIF, a LIF analogue, a LIF mimetic and a product capable of stimulating the expression of endogenous LIF, and mixtures thereof.

According to the invention, the term “culture of cells” means in particular a preparation of cells derived from a natural tissue, cultured in vitro and kept alive under artificial conditions (ex vivo, in vitro),

According to the invention, the term “library of cells” means in particular a preparation of cells treated in order to be conserved for the purpose of subsequent use. The cells may, for example, be frozen and conserved in the form of aliquots. The cells may originate from natural tissues and may be either directly stored, for example in frozen form, or amplified in vitro before being stored.

According to the invention, the expression “preparation of human keratinocytes” means in particular a set of keratinocytes obtained from a skin sample and/or from any other possible sources of skin stem cells and/or of progenitors capable of generating epidermis and/or all or some of the skin appendages.

As an alternative source of said stem cells and/or progenitors, mention may in particular be made of the hair follicle.

A preparation of keratinocytes can be obtained according to conventional cell culture methods.

In particular, the following procedure, using a skin explant taken from an individual, may be employed:

the subcutaneous tissue is removed using a scalpel;

the skin sample is decontaminated by antibiotic (for example, gentamycin) treatment;

the dermis is separated from the epidermis by proteolytic treatment (for example: trypsin and dispase) and then dissection;

the dissociation of the cells is then promoted in the presence of a solution of 0.05% trypsin and 0.02% EDTA; and the effect of the trypsin is neutralized by adding a DMEM culture medium containing 10% of serum;

the cell suspension is homogenized and is then washed in culture medium for keratinocytes (KGM, Bullet kit, Clonetics Corp).

The step of multiplication of the keratinocyte preparation may be carried out in a culture medium containing LIF, a LIF analogue, a LIF mimetic or a product capable of stimulating the expression of endogenous LIF, suitable for the multiplication of said cells and for maintaining them in an undifferentiated state.

Use may, for example, be made of a semi-defined culture medium (KGM Bullet Kit, Clonetics, Cambrex Bio Science Inc.), in the absence of feeder fibroblasts, and at low density (for example, seeding at 2400 cells/cm²).

Use may also be made of the medium described by Rheinwald and Green (Cell. 6: 317-330, 1975), in the presence of irradiated fibroblasts, and at low density (for example, seeding at 2400 cells/cm²).

More generally, any culture medium comprising LIF and intended to promote the multiplication of skin stem cells and/or of epidermal progenitors, and/or for generating a stratified epidermis, will also be part of the invention.

In particular, such a culture medium may especially contain at least one mitogenic growth factor for keratinocytes [for example: epidermal growth factor (EGF) and/or keratinocyte growth factor (KGF)], insulin, hydrocortisone and an antibiotic, (for example: gentamycin, amphotericin B).

Advantageously, said medium may also comprise a pituitary extract, for example of bovine origin, epinephrine, transferrin and/or nonessential amino acids.

Said medium may or may not contain serum and may optionally be supplemented with transforming growth factor-β (TGF-β).

The effective concentration of LIF, a LIF analogue, a LIF mimetic or a product capable of stimulating the expression of endogenous LIF, or mixtures thereof, present in the culture medium for said cells may range from 0.01 pg/ml to 1 mg/ml of culture medium, preferably from 0.1 ng/ml to 100 ng/ml of culture medium, and even more preferably from 100 pg/ml to 1 ng/ml of culture medium.

If ground cellular material or a cell extract containing LIF is used, the effective concentration of said ground material or extract present in the culture medium of the step of multiplication of said cells may range from 0.01 pg/mi to 10 mg/ml of culture medium, preferably from 10 pg/ml to 100 μg/ml of culture medium, advantageously from 100 mg/ml to 1 μg/l of culture medium.

According to a preferred embodiment, the preparation of human keratinocytes is pre-enriched with undifferentiated skin stem cells and/or epidermal progenitors, in particular by rapid adhesion on an extracellular matrix component and recovery of the cells that have adhered (cells with a high capacity for adhesion, called Adh⁺⁺⁺).

The term “extracellular matrix component” denotes in particular molecules such as collagens, laminins, fibronectin or proteogylcans.

The pre-enrichment step described in WO 03/038073 (L'OREAL/CNRS) can in particular be used.

Preferably, the adhesion step is carried out on a plastic support onto which type I collagen is adsorbed; and this step is carried out at 37° C. for a period of approximately 10 min to approximately 20 min, in particular from 12 to 15 min.

In addition, the detachment step can be carried out by mild trypsinization, for example in the presence of 0.05% trypsin and 0.02% EDTA for a brief period of time, preferably less than 10 min, in particular for a period of 1 to 2 min.

According to a particular embodiment, the cell suspension is placed in culture flasks “coated” with type I collagen (solution of collagen I (Sigma Chemical Co Ltd, Irvine, UK) diluted 2-fold in PBS, deposited in the flasks for 45 minutes, and then drying after elimination of the surplus), at a density of 200 000 cells/cm². After 12 minutes, the keratinocytes that have not adhered are removed by washing in PBS buffer. The adherent cells thus selected are detached from the support by mild trypsinization (0.05% trypsin-0.02% EDTA (Biological Industries, Kibbutz Beit Haemek, Israel) for 3 to 5 minutes at 37° C.). After neutralization of the trypsin (DMEM+10% SVF), the cells are recovered, washed, and then resuspended in KGM medium.

The adherent cell fraction selected by this method represents approximately 10% of the total keratinocytes of the epidermis.

The percentage of adherent cells generally represents 5 to 20% of the cells contained in the keratinocyte preparation, in particular 10% of the cells contained in the keratinocyte preparation.

The “Adh⁺⁺⁺” population thus selected exhibits a frequency of undifferentiated epidermal progenitors and/or of clonogenic skin stem cells that is approximately 10 times greater than that of the population of more mature keratinocytes characterized by a lower capacity for adhesion (Adh^{∓}).

The enrichment process thus makes it possible to select a population of adherent cells with a high expansion potential compared with the nonadherent cells, mainly consisting of mature keratinocytes.

Said population of Adh⁺⁺⁺ adherent cells comprises undifferentiated skin stem cells and/or epidermal progenitors, in particular characterized by a long-term expansion potential and a potential to generate a pluristratified epithelium, in particular a pluristratified epidermis, and/or a skin equivalent, and/or all or some of the skin appendages.

According to a particular embodiment, an enrichment of the keratinocyte preparation and/or of the population of skin stem cells and/or of epidermal progenitors, in LIF-reactive cells, i.e., in cells exhibiting, at their surface, expression of LIF receptor(s) (cells of LIF-R⁺ phenotype) and/or in cells capable of multiplying in an undifferentiated state in the presence of LIF, may also be carried out. The selection of the LIF-reactive LIF-R⁺ cells may, for example, be carried out by means of immunophenotypic labeling and viable cell sorting by flow cytometry, using immunomagnetic beads, and/or by means of immunoselection on a support onto which anti-LIF-R antibodies are adsorbed.

Advantageously, the method according to the invention also comprises a step of cryoconservation of said multiplied cells, under conditions that promote maintenance thereof in a differentiated state.

This cryoconservation step used in particular for obtaining cell libraries, entails freezing the cells in liquid nitrogen after expansion in the presence of LIF, in order to allow storage and/or conservation of said cells for the purpose of subsequent use.

This method of in vitro multiplication in the presence of LIF according to the invention thus makes it possible to obtain a library or a culture of undifferentiated skin stem cells and/or epidermal progenitors.

This library or culture of undifferentiated skin stem cells and/or epidermal progenitors is in particular characterized in that it is enriched in cells capable of responding to the action of LIF and/or expressing, at their surface, LIF receptors (LIF-R), which cells are referred to as LIF-R⁺.

It may be in liquid form or, advantageously, in frozen form.

Any kit for producing a library or a culture of-human undifferentiated skin stem cells and/or epidermal progenitors, comprising at least (i) a preparation of human keratinocytes and (ii) a culture medium for human keratinocyte cells supplemented with an effective amount of LIF, of a LIF analogue or of a LIF mimetic, is also within the scope of the invention.

In particular said kit will comprise a medium in which the effective amount of LIF is from 0.01 pg/ml to 1 mg/ml of culture medium, preferably from 0.1 to 100 ng/ml of culture medium.

If ground cellular material or a cell extract containing LIF is used, the effective concentration of said ground material or extract present in the culture medium may range from 0.01 pg/ml to 10 mg/ml of culture medium, preferably from 10 pg/ml to 100 μg/ml of culture medium, advantageously from 100 ng/ml to 1 μg/ml of culture medium.

The invention also features a method for preparing reconstructed epidermides and/or reconstructed skin, comprising:

a) a step entailing preparing a dermis support or a dermis equivalent; and

b) a step entailing seeding a population of human keratinocytes onto said support; an effective amount of a compound selected from among LIF, a LIF analogue, a LIF mimetic and a product capable of stimulating the expression of endogenous LIF being added to the medium, in either of steps a) and b), or both.

According to an alternative, the method for preparing reconstructed epidermides and/or reconstructed skin comprises:

a) a step entailing preparing a dermis support or a dermis equivalent; and

b) a step entailing seeding a population of undifferentiated skin stem cells and/or epidermal progenitors, cultured in the presence of a compound selected from among LIF, a LIF analogue, a LIF mimetic and a product capable of stimulating the expression of endogenous LIF, and mixtures thereof, onto said support.

In particular, said dermis support or dermis equivalent will be selected from collagen/fibroblast lattices, a dermis de-epidermalized beforehand, and artificial membranes.

By way of example of a preparation of dermis equivalent, mention may be of the protocols described in (EP-A-285,471, EP-A-285,474, EP-A-789,074, EP-A-502,172, EP-A-418,035, WO-A-9116010, EP-A-197,090, EP-A-20,753, FR-A-2,665,175, FR-A-2,689,904) or, preferably, the protocol described by Asselineau et al., 1987 (Models in dermato., Vol. III, Ed. Lowe & Maibach, 1-7).

As examples of protocols for preparing epidermis equivalents and/or skin equivalents, mention be made of those described in EP-285,471, EP-285,474, EP-418,035, WO-A-9002796, WO-A-9116010, EP-197,090, EP-20,753, FR-2,665,175, FR-2,689,904.

Very generally, reconstructed skin models consist of human keratinocytes placed on a support, often a dermis equivalent, and cultured under conditions such that they enter into a program of differentiation that results in the formation of an epidermis equivalent.

It is also possible to integrate other cell types, such as Langerhans cells (EP-0-789,074) or melanocytes, so as to reconstitute an epidermis and/or a skin similar to the natural tissues.

Any kit for producing epidermis equivalents and/or skin equivalents, comprising at least an effective amount of a compound selected from among LIF, a LIF analogue, a LIF mimetic and a product capable of stimulating the expression of endogenous LIF, and mixtures thereof, is also within the scope of the invention.

In particular, this kit may comprise (i) a dermis support or dermis equivalent, (ii) a keratinocyte preparation and (iii) an effective amount of a compound selected from among LIF, a LIF analogue, a LIF mimetic and a product capable of stimulating the expression of endogenous LIF, and mixtures thereof.

The LIF, LIF analogue, LIF mimetic or a product capable of stimulating the expression of endogenous LIF, and mixtures thereof, may be introduced alone or as a mixture into a culture medium, said culture medium being suitable for the multiplication and/or differentiation of keratinocytes.

As described above, such a medium may in particular contain at least one mitogenic growth factor for keratinocytes [for example: epidermal growth factor (EGF) and/or keratinocyte growth factor (KGF)], insulin, hydrocortisone and an antibiotic (for example: gentamycin, amphotericin B).

Advantageously, said medium may also comprise a pituitary extract, for example of bovine origin, epinephrine, transferrin and/or nonessential amino acids.

Said medium may or may not contain serum and may optionally be supplemented with transforming growth factor-β (TGF-β).

The present invention features the multiple uses of the subject LIF and of the undifferentiated skin stem cells and/or epidermal progenitors amplified in the present of LIF.

In particular, this invention features the use of undifferentiated skin stem cells and/or epidermal progenitors amplified in the presence of a compound selected from among LIF, a LIF analogue, a LIF mimetic and a product capable of stimulating the expression of endogenous LIF, and mixtures thereof, in a method for screening for and/or for evaluating active agents capable of modulating the multiplication and/or the differentiation of undifferentiated skin stem cells and/or epidermal progenitors to mature keratinocytes, or to any other cell type that is part of the constitution of the skin appendages.

In particular, this screening method comprises:

culturing undifferentiated skin stem cells and/or epidermal progenitors in the presence of a compound selected from among LIF, a LIF analogue, a LIF mimetic and a product capable of stimulating the expression of endogenous LIF, and mixtures thereof, under conditions that allow their multiplication and/or their differentiation to mature keratinocytes or to any other cell type that is part of the constitution of the skin appendages, and in the presence of an active agent to be tested;

comparing the multiplication and/or the differentiation of the cells in the presence of the active agent with the multiplication and/or the differentiation of the cells in the absence of said active agent.

The present invention also features the use of undifferentiated skin stem cells and/or epidermal progenitors amplified in the presence of a compound selected from among LIF, a LIF analogue, a LIF mimetic and a product capable of stimulating the expression of endogenous LIF, and mixtures thereof, in a method for screening for and/or for evaluating active agents capable of modulating the capacity of skin stem cells and/or of epidermal progenitors to generate a pluristratified epithelium, in particular an epidermis equivalent and/or a skin equivalent.

In particular, this method comprises:

culturing undifferentiated skin stem cells and/or epidermal progenitors amplified in the presence of a compound selected from among LIF, a LIF analogue, a LIF mimetic and a product capable of stimulating the expression of endogenous LIF, and mixtures thereof, under conditions that allow them to generate a pluristratified epidermis and/or all or some of the skin appendages, in the presence of an active agent to be tested;

comparing the capacity of the cells to generate a pluristratified epidermis and/or some or all of the skin appendages in the presence of the active agent with the capacity of the cells to generate a pluristratified epidermis and/or all or some of the skin appendages in the absence of said active agent.

Another embodiment of the invention is the use of a compound selected from among LIF, a LIF analogue, a LIF mimetic and a product capable of stimulating the expression of endogenous LIF, for preparing undifferentiated skin stem cells and/or epidermal progenitors or reconstructed epidermides and/or reconstructed skin, intended to treat skin lesions.

In particular, the undifferentiated skin stem cells and/or epidermal progenitors or the reconstructed epidermides and/or the reconstructed skin are suited to regenerate, in vivo, normal skin on damaged skin or areas of damaged skin.

There are many causes of skin lesions:

accidental causes, for example skin that has been burnt over a large surface of the body or of the face (for example: individuals suffering from third degree burns);

surgical causes, for example skin that has undergone an excision for the treatment of a naevus (Kumagai et al., Ann. Plast. Surg., 39: 483-488, 1997) or of a tattoo (Kumagai et al., Ann. Plast. Sur., 33: 385-391, 1994);

genetic causes, for example skin exhibiting alterations associated with a genetic disease; mention may in particular be made of epidermolysis bullosa, characterized by a permanent deficiency in cohesion between the dermis and epidermis (Eady et al., J. DermatoL, 28: 638-640, 2001); ichtyoses, associated with a pathological thickening of the horny layer of the epidermis, such as lamellar ichthyosis (Choate et al., Hum. Gene Ther., 7: 2247-2253, 1996), X-chromosome-linked ichthyosis (Freiberg et al., Hum. Mol. Genet., 6: 927-933, 1997); Xeroderma pigmentosum, which is characterized by a deficiency of the mechanisms of excision of DNA damage induced by ultraviolet (UV) rays, the clinical consequence of which is an increase in the frequency of appearance of skin cancers (Sarasin, Mutat. Res., 428: 5-10, 1999).

The treatment of damaged skin, when the skin is damaged accidentally or subsequent to a surgical procedure, may entail:

preparing a population of human undifferentiated skin stem cells and/or epidermal progenitors from a skin explant from a normal individual (heterologous cells) or from normal areas of the individual inhibiting damaged skin (autologous cells);

multiplying said population of human undifferentiated skin stem cells and/or epidermal progenitors in the presence of LIF;

re-implanting said cells in the areas of damaged skin, either in the form of human undifferentiated skin stem cells and/or epidermal progenitors capable of generating, in vivo, a natural epidermis and/or all or some of the skin appendages, or in the form of reconstructed epidermides and/or of reconstructed skin. In particular, the cells may be injected and/or applied to the areas to be treated, whereas the reconstructed epidermides or reconstructed skin will be grafted onto the areas to be treated.

The use of the undifferentiated skin stem cells and/or epidermal progenitors amplified in the presence of LIF, or of the reconstructed epidermides and/or reconstructed skin prepared in the presence of LIF according to the invention, will thus be in particular suitable for treating burns.

The use of the undifferentiated skin stem cells and/or epidermal progenitors amplified in the presence of LIF, or of the reconstructed epidermides and/or reconstructed skin prepared in the presence of LIF according to the invention, will thus be in particular suitable for the treatment of skin lesions associated with a skin excision, in particular of surgical origin (for example: naevus, tattoo).

The treatment of the skin lesions associated with a genetic disease may in particular entail, according to a first embodiment:

preparing a population of undifferentiated skin stem cells and/or epidermal progenitors from a skin explant from an individual suffering from a genetic disease affecting the skin;

treating said cells, ex vivo, so as to restore the defective function associated with the genetic disease, in particular by transfer of the nonmutated gene using a retroviral vector;

re-implanting said treated cells in the areas of damaged skin, either in the form of undifferentiated skin stem cells and/or epidermal progenitors capable of generating, in vivo, a natural epidermis and/or all or some of the skin appendages, or in the form of reconstructed epidermides and/or of reconstructed skin. In particular, these cells may be injected and/or applied to the areas to be treated, whereas the reconstructed epidermides or reconstructed skin will be grafted onto the areas to be treated.

According to an alternative embodiment, the treatment of the skin lesions associated with a genetic disease may entail:

preparing a population of human undifferentiated skin stem cells and/or epidermal progenitors from a skin explant from a normal individual (heterologous cells);

multiplying said population of human undifferentiated skin stem cells and/or epidermal progenitors in the presence of LIF;

implanting said cells in the areas of damaged skin, either in the form of human undifferentiated skin stem cells and/or epidermal progenitors capable of generating, in vivo, a natural epidermis and/or all or some of the skin appendages, or in the form of reconstructed epidermides and/or of reconstructed skin. In particular, the cells may be injected and/or applied to the areas to be treated, whereas the reconstructed epidermides or reconstructed skin will be grafted onto the areas to be treated.

The use of the undifferentiated skin stem cells and/or epidermal progenitors amplified in the presence of LIF, or of the epidermides reconstructed in the presence of LIF and/or of the skin reconstructed in the presence of LIF, will thus be intended for treating skin lesions associated with genetic diseases such as, for example, epidermolysis bullosa, Xeroderma pigmentosum, lamellar ichthyoses and X-chromosome-associated ichthyoses.

The use of the undifferentiated skin stem cells and/or epidermal progenitors prepared from a skin explant from an individual suffering from a genetic disease, and cultured in the presence of LIF, may also be used to set up in vitro models for studying the effectiveness of gene transfer protocols.

This will in particular involve using said cells for evaluating the frequency of genetic correction (percentage of cells exhibiting transgene insertion) and/or for evaluating and quantifying the effectiveness of functional correction (for example, study of the correction of the function initially deficient in monolayer cultures and/or reconstructed epidermides and/or reconstructed skin).

In order to further illustrate the present invention and the advantages thereof, the following specific examples are given, it being understood that same are intended only as illustrative and in nowise limitative. In said examples to follow, all parts and percentages are given by weight, unless otherwise indicated.

### BRIEF DESCRIPTION OF THE DRAWINGS

FIG. 1 shows an analysis of the effect of LIF on the clonogenic growth of stem cells and/or of epidermal progenitors. Cellular clones obtained from cells at passage 1 cultured for 8 days in the absence of exogenous LIF, and then fixed and stained (A); cellular clones obtained from keratinocytes at passage 1 cultured for 8 days in the presence of exogenous LIF at 1 ng/ml, and then fixed and stained (B); semi-quantitative analysis of the density of clones obtained under control conditions and in the presence of exogenous LIF (C).

FIG. 2 shows histological analyses of a reconstructed epidermis obtained in the presence or absence of ground material from 3T3 fibroblasts depleted or not depleted of LIF. Histological section of a reconstructed epidermis obtained in the presence of ground 3T3 material not depleted of LIF (positive control) (A); histological section of a reconstructed epidermis obtained in the absence of ground 3T3 material (negative control) (B); histological section of a reconstructed epidermis obtained in the presence of LIF-depleted ground 3T3 material (experimental condition) (C).

### EXAMPLES

### Example 1

### Undifferentiated Stem Cell and/or Epidermal Progenitor Growth Optimized in the Presence of LIF

Keratinocytes were isolated from an adult skin sample (breast plastic surgery).

After removal of the subcutaneous tissue using a scalpel, the skin sample is cut up into fragments of approximately 5 mm×5 mm, and then decontaminated by antibiotic treatment [Gentamycin (Life Technologies), 3 successive baths of 10 minutes in DMEM culture medium (Life Technologies)]. In order to allow separation of the dermis from the epidermis, the sample is then subjected to proteolytic treatment [dispase (Boehringer, Roche Diagnostics)+trypsin (Gibco, Invitrogen) overnight at 4° C.]. The epidermis is then separated from the dermis by dissection. The epidermal fragments separated from the dermal tissue are placed in a solution of 0.05% trypsin-0.02% EDTA (Gibco, Invitrogen) (15 minutes at 37° C.). The preparation is stirred periodically in order to promote dissociation of the cells. The effect of the trypsin is then neutralized by adding a culture medium containing 10% serum (DMEM+10% serum). After neutralization of the trypsin (DMEM+10% serum), the cell preparation is homogenized mechanically (pipetting), and then filtered. The cell suspension is washed, and then resuspended in KGM medium (Clonetics). The cells in suspension are counted under a microscope using a Malassez cell. The viability of the samples is estimated by the Trypan blue (Life Technologies) exclusion method.

A step for enrichment in undifferentiated stem cells and/or epidermal progenitors was carried out by means of a step of rapid adhesion on a type I collagen substrate (selection of a population with a high capacity for adhesion, Adh⁺⁺⁺).

The undifferentiated stem cells and/or epidermal progenitors can in fact be separated from the more mature keratinocytes on the basis of their rapid adhesion property. This step allows the preparation to be pre-enriched with undifferentiated stem cells and/or epidermal progenitors.

The method of enrichment by adhesion on collagen is described in WO 03/038073 and scientific publication (Fortunel et al., J. Cell Sci., 116: 4043-4052, 2003).

The cell suspension is placed in culture flasks “coated” with type I collagen [the collagen is adsorbed onto the adhesion support by deposition of a liquid solution of collagen I (Sigma Chemical) diluted 2-fold in PBS, for at least 45 minutes, and then drying after removal of the surplus], at a density of 150,000 to 200,000 cells/cm². After 12 to 15 minutes, the keratinocytes that have not adhered are removed by washing in PBS buffer. The adherent cells, referred to as Adh⁺⁺⁺, thus selected are detached from the support by mild trypsinization (0.05% trypsin-0.02% EDTA (Gibco, Invitrogen) for 3 to 5 minutes at 37° C.). After neutralization of the trypsin (DMEM+10% serum), the cells are recovered, washed, and then resuspended in culture medium (in the present case, KGM medium). The fraction made up of the adherent cells, selected by this method, represents approximately 5 to 10% of the total keratinocytes of the epidermis.

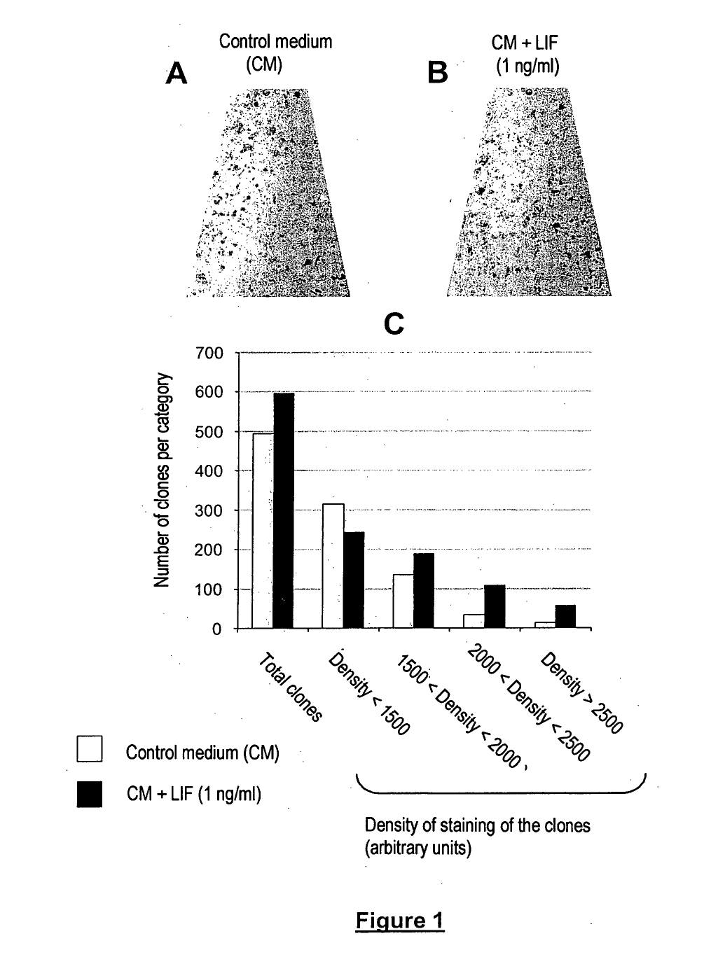

The cultures, initiated from Adh⁺⁺⁺ cells, were prepared in semi-defined culture medium (KGM Bullet Kit, Clonetics, Cambrex Bio Science Inc.), in the absence of feeder fibroblasts, and at low density (seedings at 2400 cells/cm²) in order to obtain quantifiable isolated cellular clones. At the first subculturing (passage 1), the cultures were divided into 2 batches: 1) condition identical to that described above; 2) addition of LIF at a concentration of 1 ng/ml (human recombinant LIF sold by Chemicon International Inc.). After culturing for 8 days, the cultures were fixed (70% ethanol) and stained (eosin and Giemsa) in order to analyze the characteristics of the cellular clones obtained under these 2 culture conditions (FIG. 1).

The comparative analysis of cultures performed in the absence or in the presence of LIF shows that this factor makes it possible to optimize the growth of undifferentiated stem cells and/or epidermal progenitors derived from a culture of Adh⁺⁺⁺ cells.

The macroscopic observation indicates an increase in the number of large densely-stained cellular clones (FIG. 1 A, B). The microscopic observation indicates that these clones whose development is promoted consist essentially of small cells exhibiting morphological characteristics associated with the undifferentiated state of epidermal stem cells and/or epidermal progenitors, which criterion reflects the fact that the cultures treated with LIF are “younger”. The increase in the number of large dense clones in response to LIF is confirmed by computerized image analysis, as represented in the histogram shown in FIG. 1C.

The use of LIF in a culture system therefore makes it possible to promote the multiplication of a population of human undifferentiated epidermal stem cells and/or epidermal progenitors.

### Example 2

### Positive Effect of LIF on Epidermal Reconstitution

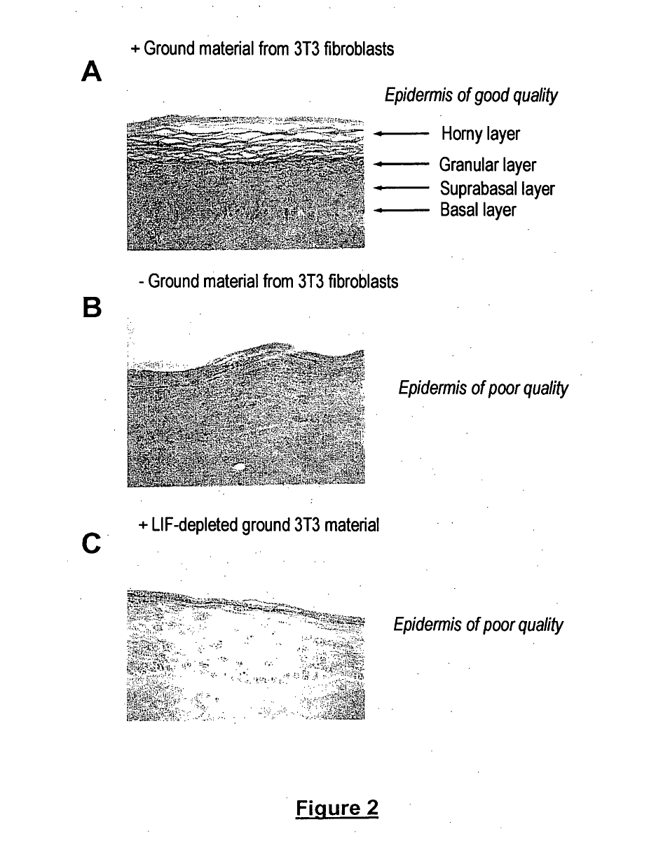

Ground material from 3T3 fibroblasts promotes epidermal reconstitution from stem cells and/or epidermal progenitors cultured in vitro and frozen. The reconstructed epidermis is of good quality: it exhibits cellular organization and stratification similar to those of a natural epidermis (FIG. 2A).

In order to test the hypothesis that LIF is involved in this property, the LIF was depleted by immunoprecipitation and then the activity of the LIF-free ground material was compared with that of the non-depleted ground material.

The LIF-depletion of the ground 3T3 material is carried out using a polyclonal antibody produced in rabbits (anti-LIF Ab, Santa Cruz, ref. SC-20087). The ground 3T3 material is incubated with the anti-LIF Ab in excess at 4° C. for several hours in order to ensure correct binding of the LIF to the Ab. The free and LIF-bound Ab is then “trapped” by adding sepharose beads onto which proteins G have been adsorbed (proteins G have a high affinity for the Fc domains of Abs). Centrifugation makes it possible to separate the beads on which the LIF is retained from the LIF-depleted ground material.

The capacity of epidermal stem cells derived from a frozen library (isolated from a mammary skin sample) to generate a reconstructed epidermis was evaluated in the presence of ground material from 3T3 fibroblasts not depleted of LIF (positive control), in the absence of ground 3T3 material (negative control), and in the presence of LIF-depleted ground 3T3 material (experimental condition). The histological characteristics of the reconstructed epidermides obtained under each condition were compared on fixed and stained sections (FIG. 2).

Whereas the organotypic cultures supplemented with ground material from 3T3 fibroblasts (not depleted of LIF) make it possible to obtain reconstructed epidermides of good quality (FIG. 2A), i.e., a cellular organization and a stratification similar to those of a natural epidermis, the cultures prepared in the absence of ground material only make it possible to obtain epidermides that do not have the required histological characteristics (FIG. 2B).

The epidermides obtained in the absence of ground 3T3 material exhibit in particular the following anomalies:

substantial intercellular vacuoles and spaces;

poor stratification;

basal layer with small number of cells;

suprabasal cells that are very stretched out;

incorrect orientation of the basal cells (parallel to the matrix);

little or no granular layer;

thin horny layer.

The cells of the experimental condition, cultured in the presence of LIF-depleted ground 3T3 material, behave in a similar manner to those cultured in the absence of ground material (FIG. 2C), which indicates that the depletion of LIF results in a loss of the beneficial properties of the ground material on the epidermal reconstruction.

Thus, the use of LIF, besides its capacity to promote the multiplication of said cells in an undifferentiated state as shown in Example 1, makes it possible to maintain the organogenic potential of said cells, i.e., their capacity to generate a reconstructed epidermis of quality, exhibiting characteristics similar to those of natural epidermis.

### Example 3

### Preparation of an Epidermis Equivalent and/or Skin Equivalent

Unless otherwise indicated, all the media and buffers used in the examples are described in Bell el al., 1979 (Proc. Natl. Acad. Sci. USA, 76: 1274-1278, 1979), Asselineau and Prunieras, 1984 (Br. J. Dermatol., 111: 219-222, 1984) or Asselineau et al., 1987 (Models in dermato., Vol. III, Ed. Lowe & Maibach, 1-7, 1987).

The dermis supports or dermis equivalents are prepared as described in Asselineau et al., 1985 and 1987 (Exp. Cell. Res., 159: 536-539, 1985; Models in dermatology, Vol. 3, pp 1-7, 1987) in the following proportions:

| | |
|---|---|
| MEM medium (1.76X) | 45% |
| Foetal calf serum: | 9% |
| NaOH (0.1N): | 5% |
| Acetic acid (1/1000): | 4% |
| Collagen: | 26% |
| Fibroblasts: | 11% |

The collagen used is type I collagen (commercial solution), but type IlI or IV collagen can also be used. It is extracted from rat tails or from calf skin by acid hydrolysis, and conserved in acidic medium at +4° C.; it polymerizes naturally by reheating to 37° C. and by decreasing the acidity (increase in pH). The collagen is dialyzed beforehand against successive baths of water+acetic acid.

The protocol is as follows: the 1.76×MEM medium in the presence of additives (1% glutamine, 1% nonessential amino acids, 1% sodium pyruvate, 1% fungizone and 1% penicillin/streptomycin), the foetal calf serum and the 0.1N NaOH are introduced into a sterile Falcon tube. Fibroblasts isolated from explants of human skin are then added at the concentration of 1.4×10⁵ cells per 1 ml of culture medium.

A volume/volume mixture of collagen in acetic acid diluted to 1/1000 is then added slowly, against the wall of the tube, so as to observe the appearance of a whitish cloud.

The entire combination is then mixed carefully and distributed into the wells of a 12-well culture plate (Costar type, reference 3512) in a proportion of 0.5 ml of mixture per cm². The culture plate is then placed in an incubator at 37° C. with 5% CO₂.

The second step entails seeding, onto said support, a preparation of keratinocytes in the presence of LIF or undifferentiated skin stem cells and/or epidermal progenitors amplified in a medium containing LIF.

According to a particular embodiment, the medium which contains the dermis equivalent is supplemented with LIF, prior to the seeding step.

The culture can then be maintained immersed in a 3F nutrient medium, which may, for example, be the medium described by Rheinwald and Green, (Cell, 6: 317-330, 1975), which medium allows keratinocyte proliferation.

After an incubation time of 3 to 15 days, preferably of 7 to 9 days, the skin equivalent is maintained by the air/liquid interface, for example by deposition on a metal screen. The liquid then preferably consists of the same nutrient medium as the previous one.

The incubation is then continued until a skin equivalent is obtained, which exhibits the characteristics of skin, i.e., the support on which there is an epidermis equivalent exhibiting the four conventional cell layer types, namely basal, suprabasal, granular and horny layers.

Thus, the incubation is continued for a period of between 5 and 30 days, preferably between 7 and 10 days.

The reconstructed skin mould thus produced comprises two entities, the support and the epidermis equivalent, which can be physically separated from one another.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for (i) promoting the multiplication, in vitro, of a population of human skin stem cells and/or epidermal progenitors while maintaining them in an undifferentiated state and/or for (ii) maintaining and/or increasing their capacity to generate a pluristratified epithelium, comprising treating same with a thus effective amount of an active agent selected from the group consisting of leukemia inhibitory factor (“LIF”), an LIF analogue, an LIF mimetic, a product capable of stimulating the expression endogenous LIF, and mixtures thereof.

2. The method as defined by claim 1, for obtaining libraries or cultures of human undifferentiated skin stem cells and/or epidermal progenitors.

3. The method as defined by claim 1, for obtaining reconstructed epidermides and/or reconstructed skin.

4. The method as defined by claim 1, said active agent comprising purified LIF, a recombinant LIF, a ground cellular material or a cell extract containing LIF, a cell culture supernatant containing LIF, or mixture thereof.

5. The method as defined by claim 1, said active agent comprising a modified LIF polypeptide or an LIF polypeptide fragment having LIF activity on skin stem cells and/or epidermal progenitors.

6. The method as defined by claim 1, said active agent comprising any agonist of the LIF receptor (LIF-R), or any active agent or cell extract capable of activating gp130 and/or the Jak/Stat and Ras/Map kinase signaling pathways.

7. A method for obtaining a library or a culture of human undifferentiated skin stem cells and/or epidermal progenitors, comprising at least one step of multiplication of a preparation of human keratinocytes in a culture medium containing an effective amount of an active agent selected from the group consisting of LIF, an LIF analogue, an LIF mimetic and a product capable of stimulating the expression of endogenous LIF, and mixtures thereof.

8. The method as defined by claim 7, wherein the preparation of human keratinocytes is pre-enriched in human undifferentiated skin stem cells and/or epidermal progenitors, by rapid adhesion on a substrate of type I collagen and recovery of the cells that have adhered.

9. The method as defined by claim 8, wherein the recovery of said cells is carried out by detachment in the presence of 0.05% trypsin and 0.02% EDTA.

10. The method as defined by claim 7, said effective amount of active agent present in the multiplication medium comprising from 0.01 pg/ml to 1 mg/ml of culture medium.

11. The method as defined by claim 7, comprising a step of cryoconservation of said cells multiplied in the presence of an active agent selected from the group consisting of LIF, an LIF analogue, an LIF mimetic and a product capable of stimulating the expression of endogenous LIF, and mixtures thereof.

12. A kit for producing a library or a culture of human undifferentiated skin stem cells and/or epidermal progenitors, comprising (i) a preparation of human keratinocyte and (ii) a culture medium for human keratinocyte cells supplemented with a thus effective amount of an active agent selected from the group consisting of LIF, an LIF analogue, an LIF mimetic and a product capable of stimulating the expression of endogenous LIF, and mixtures thereof, of between 0.01 pg/ml and 1 mg/ml of culture medium.

13. A method for preparing reconstructed epidermides and/or reconstructed skin, comprising:
a) a step which comprises preparing a dermis support or a dermis equivalent; and
b) a step which comprises seeding a population of human keratinocytes onto said support; wherein an effective amount of an active agent selected from the group consisting of LIF, an LIF analogue, an LIF mimetic and a product capable of stimulating the expression of endogenous LIF, and mixtures thereof, is added to the medium, in either of steps a) and b), or both.

14. A method for preparing reconstructed epidermides and/or reconstructed skin, comprising:
a) a step which comprises preparing a dermis support or a dermis equivalent; and
b) a step which comprises seeding a population of human undifferentiated skin stem cells and/or epidermal progenitors amplified in the presence of an active agent selected from the group consisting of LIF, an LIF analogue, an LIF mimetic and a product capable of stimulating the expression of endogenous LIF, and mixtures thereof, onto said support.

15. A kit for producing an epidermis or a reconstructed skin, comprising (i) a dermis support, (ii) a preparation of human keratinocytes and (iii) a culture medium containing an effective amount of an active agent selected from the group consisting of LIF, an LIF analogue, an LIF mimetic and a product capable of stimulating the expression of endogenous LIF, and mixtures thereof, of from 0.01 pg/ml to 1 mg/ml of culture medium.

16. A method for screening for and/or for evaluating active agents capable of modulating the multiplication and/or the differentiation of human undifferentiated skin stem cells and/or epidermal progenitors to mature keratinocytes, or to any other cell type that is part of the constitution of the skin appendages, comprising:
culturing human undifferentiated skin stem cells and/or epidermal progenitors amplified in the presence of a compound selected from the group consisting of LIF, an LIF analogue, an LIF mimetic and a product capable of stimulating the expression of endogenous LIF, and mixtures thereof, under conditions that allow their multiplication and/or their differentiation to mature keratinocytes or to any other cell type that is part of the constitution of the skin appendages, and in the presence of an active agent to be tested;
comparing the multiplication and/or the differentiation of the cells in the presence of the active agent with the multiplication and/or the differentiation of the cells in the absence of said active agent.

17. A method for screening for and/or for evaluating active agents capable of modulating the capacity of the skin stem cells and/or of the epidermal progenitors to generate a pluristratified epidermis and/or all or certain of the skin appendages, comprising:
culturing human undifferentiated skin stem cells and/or epidermal progenitors amplified in the presence of a compound selected from the group consisting of LIF, an LIF analogue, an LIF mimetic and a product capable of stimulating the expression of endogenous LIF, and mixtures thereof, under conditions that allow them to generate a pluristratified epidermis and/or all or certain of the skin appendages, in the presence of an active agent to be tested;
comparing the capacity of the cells to generate a pluristratified epidermis and/or all or certain of the skin appendages in the presence of the active agent with the capacity of the cells to generate a pluristratified epidermis and/or all or certain of the skin appendages in the absence of said active agent.

18. A regime or regimen for the treatment of skin lesions, comprising topically applying thereon the reconstructed epidermis and/or reconstructed skin prepared by the method of claim 13.

19. A regime or regimen for the treatment of skin lesions, comprising topically applying thereon the reconstructed epidermis and/or reconstructed skin prepared by the method of claim 14.

20. A regime or regimen for the treatment of skin burns, comprising topically applying thereon the reconstructed epidermis and/or reconstructed skin prepared by the method of claim 13.

21. A regime or regimen for the treatment of skin burns, comprising topically applying thereon the reconstructed epidermis and/or reconstructed skin prepared by the method of claim 14.

22. The regime or regimen as defined by claim 18, said skin lesions being due to a skin excision or associated with a genetic disease.

23. The regime or regimen as defined by claim 19, said skin lesions being due to a skin excision or associated with a genetic disease.

24. The regime or regimen as defined by claim 22, said skin lesions being associated with a genetic disease affecting the skin selected from among the group consisting of epidermolysis bullosa, Xeroderma pigmentosum, lamellar ichthyoses and X-chromosome-associated ichthyoses.

25. The regime or regimen as defined by claim 23, said skin lesions being associated with a genetic disease affecting the skin selected from among the group consisting of epidermolysis bullosa, Xeroderma pigmentosum, lamellar ichthyoses and X-chromosome-associated ichthyoses.
